(19) United States
(12) Patent Application Publication
(10) Pub. No.: US 2019/0314268 A1
(43) Pub. Date: Oct. 17, 2019

(54) Mixture of Skin Oils

(71) Applicant: Clarence McGill, Richmond, VA (US)

(72) Inventor: Clarence McGill, Richmond, VA (US)

(21) Appl. No.: 15/955,517

(22) Filed: Apr. 17, 2018

(51) Int. Cl. A61K 8/92; A61K 8/67; A61Q 19/00

(52) U.S. Cl. CPC A61K 8/922; A61K 8/925; A61K 2800/5922; A61K 8/678; A61Q 19/00; A61K 8/671

(57) ABSTRACT

Provided is a mixture of skin oils, that includes white oil, unrefined avocado oil, wheat germ oil, lanolin, and vitamins E, A and D. The mixture of natural enhancement of white oil (USP), as applied to the body, such that the added ingredients upgrade the softening and moisture-retaining characteristics inherent in the basic oil. Natural fragrances may be added. Rancidity does not occur over an indefinite period of time.

### I. TECHNICAL FIELD

The present invention relates generally to the mixture of skin oils. In particular, the present invention relates to a mixture of natural enhancement whiteoil for skin.

### II. DESCRIPTION OF THE DRAWINGS

FIG. 1 is a chart disclosing the ingredients for the mixture of skin oils that can be implemented within one or more embodiments of the present invention.

The drawing is only for the purpose of illustrating preferred embodiments and are not to be construed as limiting the disclosure. Given the following enabling description of the drawing, the novel aspects of the present disclosure should become evident to a person of ordinary skill in the art. This detailed description uses numerical and letter designations to refer to features in the drawings. Like or similar designations in the drawing and description have been used to refer to like or similar parts of embodiments of the invention.

### III. DETAILED DESCRIPTION OF THE EMBODIMENTS

As required, detailed embodiments are disclosed herein. It must be understood that the disclosed embodiments are merely exemplary of various and alternative forms. As used herein, the word “exemplary” is used expansively to refer to embodiments that serve as illustrations, specimens, models, or patterns. The figures are not necessarily to scale and some features may be exaggerated or minimized to show details of particular components.

In other instances, well-known materials, or methods that are known to those having ordinary skill in the art have not been described in detail in order to avoid obscuring the present disclosure.

The present invention discloses a mixture of natural enhancement of white oil (USP) as applied to the body, such that the added ingredients upgrade the softening and moisture-retaining characteristics inherent in the basic oil. According to an embodiment of the present invention, natural fragrances may also he added. Rancidity does not occur over an indefinite period of time.

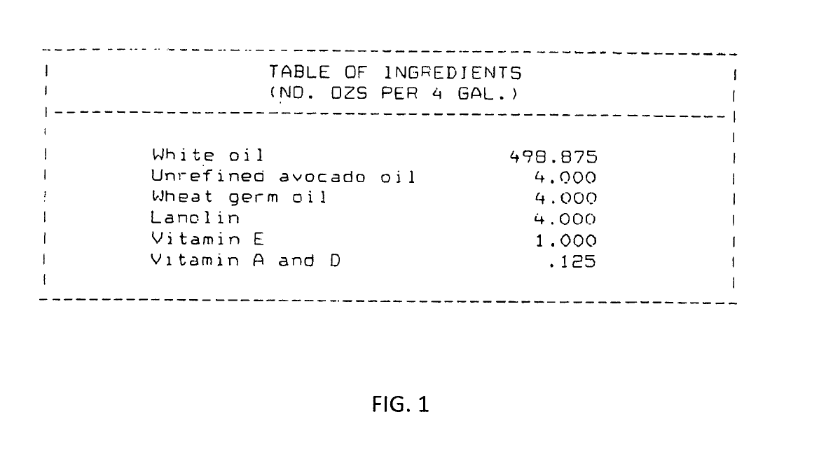

According to an embodiment of the present invention, the mixture of skin oils can be a monovin oil mixture. The advantages of monovin oil mixture is that it is non-toxic and non-irritating to both skin (dermal and epidermal) and the eyes of the user. As shown in FIG. 1 the mixture of oils includes white oil (USP), unrefined avocado oil, wheat germ oil, lanolin, and vitamins E, A and D. The composition of the skin oils mixture as shown in FIG. 1, is as follows: white oil 498.875; unrefined avocado oil 4.000; wheat germ oil 4.000; lanolin 4.000; Vitamin E 1.000; and Vitamin A and D 0.125, per 4 gallons (gal).

When one ounce of fragrance is added, white oil is reduced by one ounce.

The mixture retains moisture in the epidermal layer, and acts as a moisture barrier at the dermal layer. When applied to wet skin, the effect is softening, smoothing, and soothing, thereby resulting in a higher touch ability factor important to human interaction. Another result is the removal of dry or lightly scaled skin.

The mixture of oil is high in vitamin E, which promotes healing. Wheat germ oil is primarily polyunsaturated, adding a characteristic of absorbency to the mixture. Avocado oil is rich in vitamin A which adds in the repair of tissue and helps maintain smooth, soft and disease-free skin. Lanolin is used as a moisture barrier in various ingredient mixtures.

Vitamins A and E are added to the mixture in a pure form to augment those inherent to added oils. Vitamin D contained in combination with the Vitamin A is an incidental ingredient although it may provide a minimal counteraction to the irradiation effects of the sun on the skin. White oil's moisture also has barring characteristics at the dermal layer. Its gentle epidermal absorbency makes it a host oil in which to mix to other ingredients.

According to embodiments of the present invention, the mixture of oils has a pH value of approximately 6.8.

This written description uses examples to disclose the invention including the best mode, and also to enable any person skilled in the art to practice the invention, including performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A mixture of skin oils comprising:
white oil (USP);
unrefined avocado oil,
wheat germ oil,
lanolin, and
vitamins E, A and D.

2. The mixture of skin oils of claim 1, wherein the white oil is in an amount of 498.875 ounces; the unrefined avocado oil is an amount of 4.000; the wheat germ oil is in an amount of 4.000; the lanolin is in an amount of 4.000; the vitamin E is in an amount of 1.000; and the Vitamin A and D is in an amount of 0.125, per 4 gallons.

3. The mixture of skin oils of claim 2, further comprising a fragrance wherein the white oil (USP) is reduced by one ounce.
